(19) United States
(12) Patent Application Publication
(10) Pub. No.: US 2024/0296485 A1
(43) Pub. Date: Sep. 5, 2024

(54) BILLING DATA AGGREGATION AND FORECASTING SYSTEM

(71) Applicant: Hansei Solutions, LLC, Franklin, TN (US)

(72) Inventors: Erin Burke, Franklin, TN (US); Brittany Hines, Ashland City, TN (US); Andrew Pierno, Los Angeles, CA (US)

(73) Assignee: Hansei Solutions, LLC, Franklin, TN (US)

(21) Appl. No.: 18/178,438

(22) Filed: Mar. 3, 2023

(51) Int. Cl. G06Q 30/04; G16H 40/20

(52) U.S. Cl. CPC G06Q 30/04; G16H 40/20

(57) ABSTRACT

Some implementations of the disclosed systems, apparatus, methods and computer program products are configured for implementing billing data processing systems. Such systems may receive non-standardized data from a plurality of sources and provide for the creation of data for billing and/or forecast from such non-standardized data. The system and techniques described herein aggregates non-standardized data from a plurality of sources to create standardized form modules. Furthermore, the system and techniques described herein allow for forecasting.

### BACKGROUND

Medical billing systems are extremely complicated and are generally processed in complicated and time consuming manners. Each medical payment provider (e.g., insurance company) may require different information, offer different payment terms, and respond in different timeframes. Thus, current medical billing practices are opaque, hard to understand, and with hard to predict results.

### SUMMARY

Described are methods and systems for billing data processing. In a certain embodiment, a method may be disclosed. The method may comprise receiving non-standardized procedure data from a graphical user interface (GUI) of a user device, standardizing the procedure data, wherein the standardizing comprises parsing the non-standardized procedure data to determine procedure data details and creating standardized procedure data, the standardized procedure data comprising a standard data structure, wherein the parsed procedure data details are populated within the standard data structure, determining, based on the standardized procedure data, procedure details, determining cost elements based on the procedure data, accessing a cost database to obtain the cost data indicating the cost data elements, forecasting a cost range based on the cost data elements, and communicating the cost range for display on the GUI of the user device.

In another embodiment, a system may be disclosed. The system may comprise a plurality of databases, a memory, and a processor, configured to receive instructions from the memory and perform operations comprising: receiving non-standardized procedure data from a graphical user interface (GUI) of a user device, standardizing the procedure data, wherein the standardizing comprises parsing the non-standardized procedure data to determine procedure data details and creating standardized procedure data, the standardized procedure data comprising a standard data structure, wherein the parsed procedure data details are populated within the standard data structure, determining, based on the standardized procedure data, procedure details, determining cost elements based on the procedure data, accessing a cost database of the plurality of databases to obtain the cost data indicating the cost data elements, forecasting a cost range based on the cost data elements, and communicating the cost range for display on the GUI of the user device.

Illustrative, non-exclusive examples of inventive features according to the present disclosure are described herein. These and other examples are described further below with reference to figures.

### BRIEF DESCRIPTION OF THE DRAWINGS

The disclosure may best be understood by reference to the following description taken in conjunction with the accompanying drawings, which illustrate various embodiments.

FIG. 1 illustrates a block diagram of an example system, in accordance with some embodiments.

FIG. 2 illustrates a block diagram of another example system, in accordance with some embodiments.

FIGS. 3-6 are process flowcharts illustrating various techniques or portion thereof for billing data aggregation, in accordance with some embodiments.

FIG. 7 illustrates an example of a neural network, configured in accordance with some embodiments.

FIG. 8 illustrates a machine learning billing data training technique, in accordance with some embodiments.

FIG. 9 illustrates a block diagram of an example computing system, in accordance with some embodiments.

### DETAILED DESCRIPTION

In the following description, specific details are set forth to provide illustrative examples of the systems and techniques described herein. The presented concepts may be practiced without some, or all, of these specific details. In other instances, well known process operations have not been described in detail to avoid unnecessarily obscuring the described concepts. While some concepts will be described with the specific examples, it will be understood that these examples are not intended to be limiting.

Some implementations of the disclosed systems, apparatus, methods and computer program products are configured for implementing medical billing data processing systems. In a certain embodiment, such systems may receive non-standardized data from a plurality of sources and provide for the creation of data for billing (e.g., insurance billing, such as billing for healthcare insurance) from such non-standardized data. The systems and techniques described herein may allow for the automated creation and population of data used for such billing.

Typically, billing is performed through hand population and filling out of one or more manual forms, typically with a lot of different entries that must conform to the needs of insurance providers and state and federal regulations. Each form tends to be specific to the insurance carrier and may differ from others in terms of formatting, wording, entry type (e.g., whether to provide a response in a written sentence or via a checkbox), information required, and other such aspects. Nonetheless, while the forms or specific entries may be different, the individual portions of data required may be similar or the same, leading to significant end user confusion. That is, though wording may be different, certain required portions of data may nevertheless be shared between different insurance carriers. Furthermore, electronic storage of such a vast number of forms leads to wasted memory and, additionally, places significant processing loads on systems to determine the appropriate form to select from within the database.

The system and techniques described herein, instead of utilizing stored pre-created forms, aggregates non-standardized data from a plurality of sources to create standardized form modules. For each instance, the appropriate form modules that would obtain the needed data for the situation is determined, accessed, and combined into the appropriate form for a specific submission. As such, the specific standardized entries that are needed are identified and aggregated. The systems and techniques described herein reduce memory storage requirements as only form data associated with the entries need to be stored. Such form data can utilized in different composite forms and can be used for a plurality of different entry types and, thus do not need to be stored multiple times due to different wording changes in different forms. Furthermore, the base forms themselves are not stored within a database, further saving memory resources. Form responses may include any type of input, including text, audio, video, images, code, and other types of responses.

Furthermore, the system and techniques described herein allow for forecasting of claims. Typically, insurance claims require a large amount of time to process and the final amount paid, if any, by the insurance company is hard to determine. The current systems and techniques allow for the aggregation of data and accurate prediction of the future value of insurance claims. Forecasting techniques are thus improved.

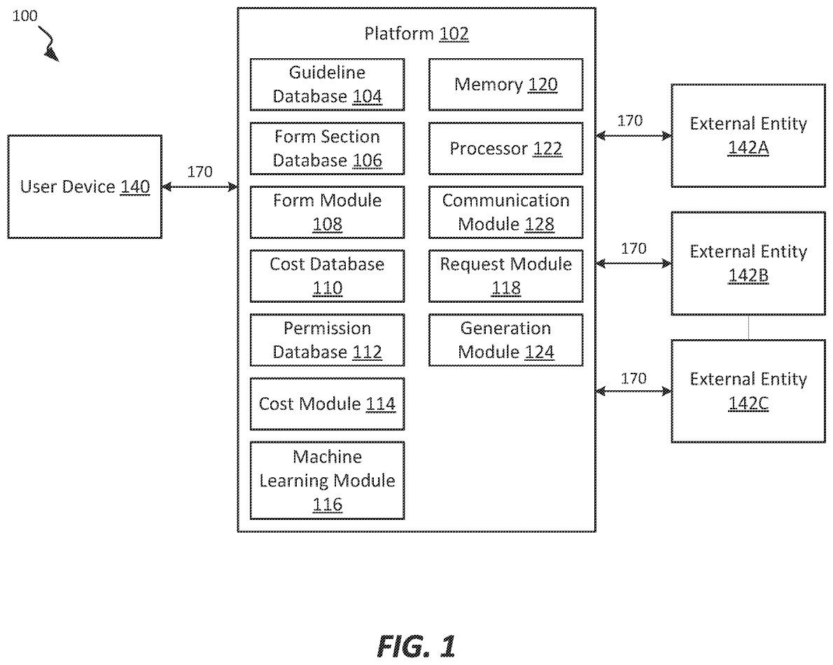

FIG. 1 illustrates a block diagram of an example system, in accordance with some embodiments. FIG. 1 illustrates system 100 with platform 102 communicatively coupled to user device 140 and external entities 142A-C. It is appreciated that, for the purposes of this disclosure, when an element includes a plurality of similar elements distinguished by a letter following the ordinal indicator (e.g., compliance entities “142A,” “142B,” and “142C”) and reference is made to only the ordinal indicator itself (e.g., “142”), such a reference is applicable to all the similar elements.

Platform 102 includes guideline database 104, form section database 106, form module 108, cost database 110, permission database 112, cost module 114, machine learning module 116, memory 120, processor 122, generation module 124, and communication module 128. Platform 102 may be configured to provide the various forms, forecasts, and other data for the techniques described herein in response to requests from user device 140. In various embodiments, user device 140 and/or external entities 142A-C may be communicatively coupled with platform 102 via communications network 170.

Variously, the current disclosure may reference certain entities. For example, “vendor” may refer to, for example, insurance vendors that provide insurance coverage and payment for procedures performed. “Service provider” may refer to a healthcare provider, whether a practitioner or entity (e.g., hospital or hospital network) that may provide services such as healthcare services.

Communications network 170 may be any wired or wireless communications connection that allows for data to be sent. Network 170 may be, for example, a wired Ethernet connection or a wireless connection such as WiFi, 3G, 4G, 5G, or another such connection that allows for data to be transmitted. In various embodiments, the various components of the systems described herein may utilize one, some, or all of such data connections to communicate and/or receive data.

User device 140 may be a user device of a user of platform 102. Such a user may be, for example, a user associated with platform 102 such as a service provider (e.g., healthcare provider such as a hospital or healthcare practice), a billing organization (e.g., an organization that aids in billing for a service provider), and/or another such user. User device 140 may be, for example, desktop computing devices, portable computing devices (e.g., laptops, tablets, smartphones, and/or other electronic devices), wearable devices, and/or other such electronic devices. In various embodiments, a plurality of user devices associated with one or more users may be communicatively coupled to platform 102. User device 140 may be configured to provide data to platform 102, such as data associated with a procedure or other service performed or a request for a composite form or forecast. In various embodiments, platform 102 may generate a graphical user interface (GUI) and communicate the GUI to user device 140 for display. The GUI may be configured to receive data from a user of user device 140. The GUI may also be configured to display data, such as the output of any techniques described herein.

Requests provided to platform 102 may be received directed by communication module 128 to request module 118. Request module 118 may be configured to receive requests (e.g., requests for reimbursement, forecasting, and/or other services) from user device 140. Such requests may result in platform 102 accessing data of various database and creating an output.

In various embodiments, request module 118 may be configured to receive such requests and standardize the results. Thus, for example, the various user devices 140 and, indeed, different requests provided by the same user device 140, may provide different requests in a different non-standardized formats. For example, a first request may be communicated via e-mail, a second request may be communicated via a request for reimbursement form, and a third request may be communicated via a data communicated from a web portal that receives data via a graphical user interface (GUI). Request module 118 may receive data from such different sources and convert the data into standardized requests. For example, standardization may include converting the data specifying the requirements to a standardized form. Standardization may also include parsing audio, video, or images to extract data. Request module 118 may be configured to parse the request and determine the specific requests (e.g., via machine learning, text recognition, data or metadata of the request, third parties, and/or another such techniques). Such standardization breaks down the requests into specific requirements. The requirements are then converted into standardized data (e.g., data markers such as alphanumerical, phrase based (e.g., a phrase indicating the data required), and/or other markers that indicate the category of the request and/or parties involved within the request, the request type, the amount, the conditions behind the request (e.g., markers indicating that the request is associated with a procedure or an exam), and/or other aspects of the request. Request module 118 may then accordingly store such data for the requests that have been converted into the standardized form.

External entity 142 may be electronic devices of various entities, such as vendors. Such vendors may be, for example, insurance companies, other service providers, patients, requirements database, legal databases, regulatory bodies, government entities, and/or other such entities that may be configured to provide data to and/or receive data from platform 102. Such data may include, for example, data directed to regulations to be complied with, the requirements of various insurance companies and other parties, various forms for specific situations (e.g., in non-standard formats indicating the data that is required for various situations), and/or other such data. In various embodiments, data provided by external entity 142 may, thus, be non-standard data that, for example, may only be useful for use in the situation circumstance and/or with the specific external entity. Accordingly, data provided by a certain external entity 142 may not be compatible for use with other external entities.

Guideline database 104 may be a database configured to store the requirements of various external entities 142. Thus, for example, in order to properly process claims, an insurance vendor may require a certain set of information from the service provider. The set of information may differ between different insurance vendors. Guideline database 104 may be configured to store data directed to the requirements of different insurance vendors and/or other such vendors.

In certain embodiments, guideline database 104 may be configured to interpret guidelines (e.g., the requirements of an insurance vendor, law making entity, regulation making entity, and/or another such entity). Such interpretation may be through, for example, machine learning according to the techniques described herein. Guideline database 104 may, for example, receive text associated with such guidelines. Through OCR or another text recognition technique, guideline database 104 may parse the text of the guidelines, determine keywords or phrases within the guidelines (e.g., keywords or phrases associated with certain requirements and/or keywords or phrases indicating deliverables for the requirements). Such recognition and parsing may then provide for the interpretation of the respective guidelines. In various other embodiments, guideline database 104 may, alternatively or additionally, receive inputs such as audio or video files, and then interpret and parse the instructions in such files (e.g., through voice recognition).

In certain embodiments, guideline database 104 may be configured to standardize data received from various external entities and store the standardized data within guideline database 104. Thus, for example, guideline database 104 may be configured to determine the requirements of various external entities 142 (e.g., via machine learning, text recognition, metadata of files provided by the external entity, third parties, and/or another such techniques) and standardize such requirements. Accordingly, for example, such standardization may include converting the requirements to a standardized form. Thus, though different requirements from different external entities 142 may include different wording, the standardization technique breaks down the requirements into the specific data that is actually required to be obtained. Such requirements are then stored as standardized data that may be interchangeably utilized for different user and/or external entities.

Guideline database 104 may be configured to store data in one of a plurality of different manners. Thus, for example, guideline database 104 may assign identifiers to each piece of information required and may include a data table calling out the identifiers associated with the guideline for each vendor/category. In certain embodiments, such identifiers may be alphanumerical, phrase based (e.g., a phrase indicating the data required), links or other connections to the form section required (e.g., linking to the appropriate form section stored within form section database 106), and/or through another technique indicating the appropriate data required for a form. Based on the identifiers, the different types of information that is required for each vendor/category may be determined. Form section database 106 may then be accordingly accessed to retrieve the form sections that correspond with the information.

Form section database 106 may be a database configured to store various form sections. Form sections may be individual sections that are configured to receive input to provide a data element required by a vendor. Thus, form sections may be data elements that identifies the data entry required. One or more forms sections may be combined to create a complete form, which may be provided to a service provider for execution (e.g., completion of the form). Forms that have had their various form sections completed (e.g., with the requisite data provided to the form sections) may then be provided to a vendor. When the form sections are properly completed, the forms may meet the information requirements of the vendor.

In various embodiments, the form section may be configured to allow a user to provide needed data. The form section may be configured to allow for data to be provided through one or more different techniques. For example, in certain embodiments, certain form sections may be configured to receive natural language (e.g., the user may provide responses in words, phrases, and/or sentences), multiple choice selections (e.g., one or more pre-determined responses), alphanumerical responses (e.g., on a scale of 1-10), and/or other such responses. Other embodiments, of form sections may be configured to interface with the user device or a database associated with the user and automatically determine (e.g., from data or metadata stored on the user device and/or within the database) the response to the form section and generate the appropriate data.

Form module 108 may be a module configured to create the appropriate form for the vendor and/or request. Accordingly, form module 108 may identify the request (e.g., insurance reimbursement request), the associated vendor, and other associated parties (e.g., from request module 118) and receive data from guideline database 104 for the appropriate vendor and request. Based on the data received from guideline database 104, form module 108 may then determine the appropriate composition of the form that is required for the vendor and/or the request. Form module 108 may then access form section database 106 to obtain the appropriate form section and generate the appropriate form.

As described herein, form module 108 may include instructions, algorithms, rules, and/or other data transformation techniques where inputs are provided to form module 108 and transformed to outputs (e.g., a form). Such instructions in form module 108 may be updated based on, for example, changes to data within guideline database 104, form section database 106, based on requests received from request module 118, feedback from users and/or other entities, and/or other such updates. Thus, the techniques for updating the forms may be continuously updated.

Generation module 124 may be configured to generate completed forms with the data required (e.g., by various entities or vendors). Thus, for example, form module 108 may output a form that includes the appropriate form section and provide such a form to generation module 124. Generation module 124 may then access the appropriate databases to “fill in” the portions of the form to provide a completed form.

In various embodiments, the separation of form module 108 and generation module 124 provides for data safety and more consistent form generation as form module 108 is not tasked with filling in the forms that it generates. Accordingly, the forms generated by form module 108 is agnostic as to whether platform 102 includes the needed data for completing the form, ensuring that the forms generated by form module 108 are solely generated to meet the requirements of the requests. Furthermore, separation of form module 108 and generation module 124 allows for the various algorithms, rules, and/or other data transformation techniques of the various modules to be specialized and, thus, optimized.

Cost database 110 may be configured to store cost data associated with various vendors and/or requests. Such data may include, for example, for a given vendor and request, the typical response provided by the vendor, the average or range of (e.g., the one standard deviation range) reimbursement provided by the vendor, the average or range of time required to provide payment, and/or other such data. The data may be reported by various external entities 142, by various users, through access of external databases (e.g., databases that store data related to reimbursement percentage, amount, and/or time of payment for a vendor), through raw data that is then analyzed, and/or through other such techniques. In certain embodiments, the data may be associated with and/or divided based on various categories (e.g., the data provided by the form may specify a plurality of categories, and specific cost data may be associated with each of the categories). As such, cost database 110 may be a database that includes entries that are categorized based on vendor, request, reimbursement amount, reimbursement timeframe, and/or other such aspects. In various embodiments, data stored within cost database 110 may be non-standard or standardized. Non-standard data may, when utilized in the techniques described herein, be standardized through the various techniques described herein to allow for an accurate comparison between different data sets.

For example, cost database 110 may be configured to store billing data records, compensation data records, practitioner financial records, and/or other such records. Billing data records may include data directed to bills that are provided to clients and/or for compensation (e.g., the amount billed by the practitioner, provided by the practitioner billing). Compensation data records may include data directed to the amount of compensation provided to the biller in response to billing requests (e.g., replies to invoices provided by, for example, the payment provider). Practitioner financial records may include data directed to the financial performance of an entity and/or service provider. As an illustrative example, billing data records and compensation data records may be of a non-standard form while practitioner financial records may be of a standard form. Non-standardized data may be standardized before being used in the techniques described herein.

Permission database 112 may be configured to store data directed to various access levels of users, vendors, and/or requests. Permission database 112 may include data indicating whether a certain user is able to obtain certain form sections (e.g., form sections may include tiered permission levels), access to form module 108, and/or other such permission levels. Permission database 112 may also include data indicating whether a user can access the data of certain vendors and/or requests. The techniques described herein may include, explicitly or implicitly, a step where data from permission database 112 is accessed to determine whether the requesting user has the appropriate permission to request the technique.

Cost module 114 may be configured to provide forecasts of the likely compensation of an action being performed. Cost module 114 may perform such forecasts according to the techniques described herein and utilizing, for example, data from cost database 110 and/or other such databases.

Machine learning module 116 may be configured to perform machine learning of various techniques described herein. For example, machine learning module 116 may be a neural network and/or another module configured to receive data and provide outputs. Thus, for example, machine learning module 116 may be trained to receive a request, access the data of various databases, and provide the appropriate output (e.g., creating the appropriate form from various form sections).

In various situations, vendors, requests, and/or other such aspects may utilize different language to describe the same or similar data requirements. Conversely, based on the context (e.g., different regulatory bodies, subject matter, etc.) similar language for two requirements may actually describe different requirements. Machine learning module 116 may be configured to receive data utilizing any such format, diction, and/or other aspects that are different and determine the requirements in a standardized format, based on training.

Memory 120 may be any type of memory device configured to store data and/or instructions. Memory 120 may be, for example, a harddrive, a solid state device, and/or random access memory (RAM) and may include transitory or non-transitory computer-readable media. Memory 120 may be configured to store instructions for performing the techniques described herein. Such instructions may be communicated to various elements of compliance platform 102, such as processor 122.

Processor 122 may be a single or multi-core processor. As described herein, processor 122 may be configured to perform operations of platform 102, as described herein. Processor 122 may be any type of single or multi-core processor that allows for electronic data processing. It is appreciated that processor 122 may perform the techniques described herein utilizing one or more databases, modules, and/or other system components as described herein. Accordingly, processor 122 may perform the techniques described herein while calling upon the databases, modules, and/or other system components. In certain embodiments, such modules and/or other system components may be called by processor 122 as one or more APIs.

Communication module 128 may be configured to communicate with various external devices, via communications network 170. Thus, for example, communication module 128 may be configured to communicate with user device 140 and/or external entities 142 (e.g., receive and/or provide data to such elements). Such data may be utilized to perform the techniques described herein.

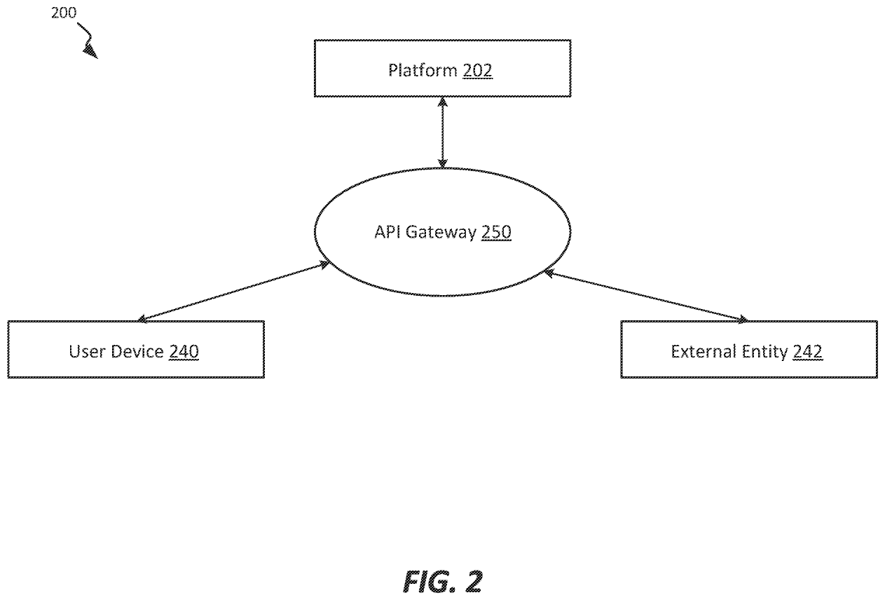

FIG. 2 illustrates a block diagram of another example system, in accordance with some embodiments. FIG. 2 illustrates system 200, which is configured to implement the techniques and processes described herein. System 200 may include platform 202, API gateway 250, user device 240, and external entity 242.

User device 240 and external entity external entity 242 may be as described herein. One or more of user device 240 and external entity 242, may be configured to provide data to and/or access the various databases, modules, and other components of platform 202 via API gateway 250. Thus, the various techniques of platform 202 may be configured as one or more APIs and API gateway 250 may provide access to such APIs. For example, API gateway 250 may receive log-in or authentication credentials from a user of user device 240. API gateway 250 may verify that the requesting entity includes the appropriate permission to access the requested API. Based such credentials, API gateway 250 may then allow for user device 240 to access an API of platform 202 (e.g., an API providing for the creation of one or more forms from constituent form sections).

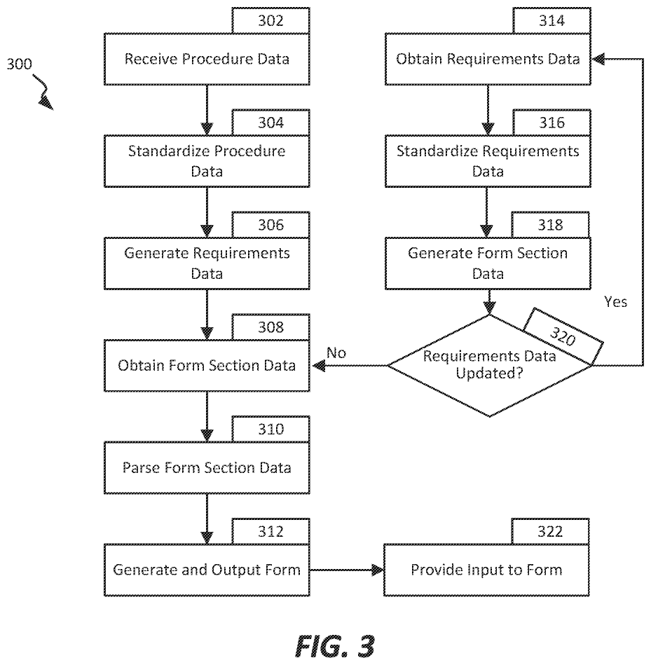

The various systems and techniques (e.g., access to databases and/or modules) described herein may be provided as one or more APIs. Thus, for example, each of the techniques of FIGS. 3-6 may utilize one or more separate APIs. Furthermore, in certain embodiments, portions of techniques, such as accessing one or more databases, may be separate APIs. Thus, authentication of a user and determination of the access privileges of the user may be provided by one API while physical access to the database may be provided as a separate API. Such a configuration allows for individual techniques described herein, or portions thereof, to be utilized by third parties. Thus, platform 102 may create forms requested by users through the calling of just the APIs related with the action.

FIGS. 3-6 are process flowcharts illustrating various techniques or portion thereof for billing data aggregation, in accordance with some embodiments. FIG. 3 illustrates technique 300, which illustrates a technique for generating forms with the systems described herein.

In 302, procedure data may be received from one or more vendors and/or service providers. The procedure data may be received as input from a GUI presented on a user device. The procedure data may be associated with the vendor (e.g., the request for reimbursement may be a request for reimbursement from the vendor) and/or service provider (e.g., the service provider may be the party that performed the service for which reimbursement is requested) and one or more requests (e.g., insurance reimbursement requests). The procedure data may identify or be associated with one or more categories, such as services performed (e.g., one or more medical procedures performed). In certain such embodiments, such identification or association may be determined from metadata of the procedure data provided. In other embodiments, such identification or association may be determined from other data of procedure data (e.g., the procedure data may include a data entry that may be analyzed by platform 102.)

In various embodiments, procedure data received in 302 may be of a non-standard format. That is, platform 102 may be configured to receive procedure data from a plurality of different sources and such data may be of different formats, as described herein. In 304, the procedure data received may be converted to a standardized format, according to the techniques described herein. In certain embodiments, standardization may include anonymization or encryption of such data. Such anonymization may include, for example, blurring specific values (e.g., numerical values, names, voices, videos, and/or other such data) of the data. Such blurring may include, for example, changing a specific instance to a value range, blurring of certain portions of a video, changing a pitch of a voice, and/or other such techniques. Encryption may include, for example, homomorphic encryption and/or another such encryption technique.

In 306, procedure data is analyzed by platform 102 and requirements data is generated from analysis of the procedure data. Analysis of the standardized procedure data may allow for the determination of the vendor that reimbursement is requested for and the category and specifics for the vendor. Thus, for example, the same procedure may be categorized under different reimbursement categories by different vendors. Furthermore, different vendors may include cut-offs and threshold depending on the amount of reimbursement requested, the timetable, the type of patient, the association between the vendor and the service provider, and/or other such categories. Analysis of the standardized procedure data may allow for the determination of such details and such details may then be used to generate the requirements data by, for example, accessing the appropriate data from guideline database 104.

Requirements data may indicate the data that is required by the vendor, based on the details, in a submitted request in order for the request to be accepted and/or granted by the vendor. In various embodiments, requirements data may include requirements for a plurality of different requests (e.g., separate services performed). Requirements data may include, for example, the data elements that need to be submitted to the vendor. Such data elements may be required for the request to be accepted and/or granted. The lack of such data elements may, therefore, result in the vendor rejecting such requests.

In various embodiments, requirements data may include, for example, identity data (e.g., identifying the service provider, vendor, request, category of request, and/or other such identifying information), data associated with the performance of actions associated with the request (e.g., data indicating the date, time and/or procedure performed, details of the performance of the procedure, identity of individuals associated with the procedure, the amount of time and resources that were spent, and/or other such aspects), administrative data (e.g., data identifying the department that the request should be provided to), reimbursement data (e.g., billing amount and payment terms), and/or other such data. Requirements data may be determined from, for example, the data within guideline database 104. Thus, platform 102 may receive the procedure data in 302, receive and/or identify the service provider, vendor, and/or request in 306, and access guideline database 104 to determine the requirements data.

In 308, based on the determined requirements data, the appropriate form section data may be obtained. Thus, for example, the requirements data informs the data elements that are required to be submitted. Variously, requirements data may be associated with one or more items of form section data. For example, each requirement within requirements data may be associated with one or more form sections. Such form section(s) may be form section(s) that are configured to obtain the data needed to fulfill the requirement. Accordingly, based on the requirements data indicating the various requirements, the associated form section data may be obtained from the form section database. The form section data may be data as described herein. Accordingly, form section data associated with one or more sections of a form may be obtained in 308 and received as inputs.

In 310, the obtained form section data may be analyzed to determine whether the form section is appropriate for obtaining the target data as specified by the requirements data. In certain embodiments, responses to the form section data may be obtained, whether manually or automatically, in 310. Thus, for example, platform 102 may obtain the form section data, parse the form section data to determine the data that the form section is configured to obtain, and determine whether one or more databases within platform 102 or communicatively coupled to platform 102 includes the required data in the form section. If the data is stored within one or more such databases, the data may be obtained and appended onto the appropriate form section (e.g., the form section may be filled out). Otherwise, in certain embodiments, the form section may be communicated to the user device to obtain such data. In various embodiments, such form sections may be included as part of the assembled form (in 312) and may be communicated to the user (in 322).

In 312, the form may be generated from the various form sections obtained in 308. The form may, thus, be a combination of the various form sections, as well as other data. The generated form may then be output (e.g., communicated) in 312. In various embodiments, if the various form sections of the form has obtained the needed data from the various databases (e.g., by one or more modules of platform 102), the form may be automatically completed in 322 and communicated directly to the vendor. Thus, for example, the generated form (e.g., generated by form module 108) may be used as input for one or more other modules (e.g., generation module 124) of platform 102. Generation module 124 may then obtain the needed data from the various databases and generate and output a form with the needed data filled in.

In certain other embodiments, the form may be communicated to the user (e.g., the user device, for display on a GUI of the user device). The user may then provide the completed form or data for competing the form to platform 102. Such data may be of a format usable for platform 102 to complete the form. Thus, for example, platform 102 may receive such data, analyze the data to determine the category and/or form section associated with the answer (e.g., based on the data provided and/or the metadata, such as metadata associating the user's response to that of the respective form section by, for example, metadata indicating which part of the generated form the response is provided for.)

Certain embodiments of technique 300 may, alternatively or additionally, determine and/or create the form section data. Such new form sections may be based on the requirements of one or more vendors. For example, in 314, requirements data may be obtained from one or more vendors. Such requirements data may indicate data that is required for one or more requests to be accepted and/or approved by a vendor, and may be obtained per the techniques described herein.

As the requirements data may be non-standard (e.g., different vendors may provide requirements data in different formats), the requirements data received in 314 may be standardized in 316. Standardization may be according to the techniques described herein. The standardized requirements data may allow for

In 318, form section data may be determined and/or created. Thus, for example, form sections may be created based on the standardized requirements data. Creation of the form section data may be via machine learning, specific rules (e.g., previously configured textual inquires with the needed information inserted into a blank space), multiple choice questions where the selections are determined from one of a plurality of possibilities (e.g., the standardized requirements data may require selection of one of four possible practice groups for a given procedure and such data may be obtained through a multiple choice question), audio, video, or images, manually, and/or through another such technique. In certain embodiments, the form module 108 may be configured to determine the appropriate format for a specific form section (e.g., the question format type). Accordingly, such form sections may be configured to obtain the data that is specified in the requirements for the section (e.g., within the requirements data).

In 320, form section data may already have been created in 318 and platform 102 may determine whether requirements for vendors and/or requests have been updated. For example, vendors may change the data required to be submitted for acceptance and/or approval, whether periodically or irregularly. Regulatory bodies may also update the data that is required. Regulation updates may affect the data that is required as well.

If platform 102 determines that an update is required, technique 300 may return to 314 from 320. In certain embodiments, an update to any regulation or requirement may automatically trigger technique 300 to return to 314. Other embodiments may analyze updates (e.g., through natural language processing, machine learning, and/or through other techniques) and determine whether an update is actually required. If platform 102 determines that no update is required, the form section data may not be updated and may accordingly be utilized in the rest of technique 300.

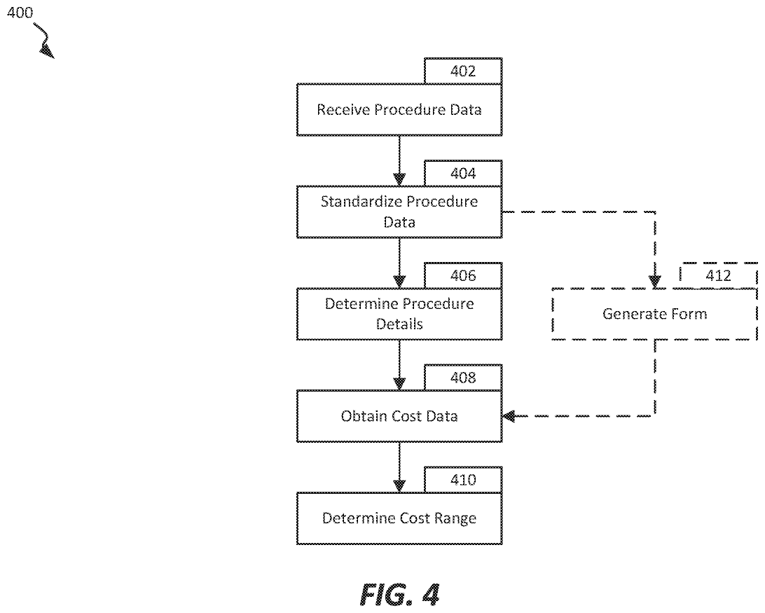

FIG. 4 illustrates technique 400, which may be a technique for estimating the likely payment of a request. Thus, technique 400 may be a technique for forecasting the likely value of, for example, a current insurance claim. As insurance claims require a significant period of time for processing, the actual value of a claim is often unclear when the claim is submitted. Technique 400 allows for the forecasting of such value, which provides for significantly greater clarity as to the expected future cashflow for an organization. Alternatively or additionally, technique 400 may be utilized for forecasting of other items.

In 402, procedure data may be received. Procedure data may be data generated by an action performed by the user. Thus, for example, procedure data may be data generated by a user and may be associated with a request. The user may have, for example, performed a service such as a medical procedure and the request may be a request for compensation (e.g., from an insurance company). The procedure data may include data associated with the service provider, the service receiver, the services provided (including various details of the services provided), the entity (e.g., hospital) that the service provider is associated with, the vendor that the request is directed towards, the request (e.g., requested amount), and/or other such information. In various embodiments, some or all of the procedure data may be provided as part of a request to the vendor.

Platform 102 may be configured to receive procedure data from a plurality of sources. The procedure data may be different formats. In 404, procedure data may be standardized. Such standardization may be according to the techniques described herein. Thus, for example, the procedure data received may first be analyzed to determine the various data elements and the information contained within the data elements. Thus, the data elements of the procedure data may be analyzed to determine the service provider, the service receiver, the services provided, the entity that the service provider is associated with, the vendor that the request is directed towards, the request, and/or other such information.

Upon determination of such data elements, the data shape of the procedure data may then be converted into a standard data shape. Accordingly, the procedure data may be converted into a standardized format (e.g., a data shape that includes data elements corresponding to one, some, or all of the information contained within the procedure data). In certain instances, the procedure data may not include certain data elements of the standardized format. In such instances, nominal information may be provided or such data elements may be blank. In certain embodiments, data elements that are not used for standardization and/or used to determine other data to access may be anonymized and/or encrypted as part of the standardization process and/or later in the process. Such anonymization may include, for example, blurring specific values (e.g., numerical values, names, voices, videos, and/or other such data) of the data. Such blurring may include, for example, changing a specific instance to a value range, blurring of certain portions of a video, changing a pitch of a voice, and/or other such techniques. Encryption may include, for example, homomorphic encryption and/or another such encryption technique.

In 406, the standardized procedure data is analyzed to determine procedure details. Thus, for example, platform 102 may analyze the procedure data to determine the service provider, the service receiver, the services provided, the entity that the service provider is associated with, the vendor that the request is directed towards, the request, and/or other such information. Analysis of the procedure details may, in certain embodiments, be limited to the data communicated or to be communicated to the vendor. However, other embodiments, may analyze additional procedure details, including details not communicated to the vendor.

Based on such information, in 408, platform 102 may access the appropriate database (e.g., cost database 110) and obtain data related to information indicated by the procedure data. Thus, for example, platform 102 may obtain cost data associated with the service provider, the service receiver, the services provided, the entity that the service provider is associated with, the vendor that the request is directed towards, the request, and/or other such aspects.

Additionally or alternatively, in optional 412, a form may be generated according to the techniques described herein (e.g., the technique described in FIG. 3). Such a form may include various form sections. The form and/or the form sections may then inform the appropriate database to access in 408. Thus, for example, the various form sections of the form may include a form section identifying the service performed (e.g., a form section that requests the technical details of the services performed, which may by necessity identify the category of service), one or more form sections identifying the service provider or vendor (e.g., certain vendors may require certain form structures), a form section that identifies the jurisdiction, and/or other such details. Based on such details, the appropriate database may be accessed and searching of the database may be appropriately informed.

Thus, in the previous example, in various embodiments, cost database 110 may store information such as the amount of reimbursement and the time needed for reimbursement. Such information may be stored in, for example, a database and/or other data structure that allows for the cross referencing of the cost data of one or more reimbursements provided by the vendor. The cross referencing may be according to the various aspects as described herein (e.g., the service provider, the service receiver, the services provided, the entity that the service provider is associated with, the vendor that the request is directed towards, the service provided, the jurisdiction, the request, and/or other such aspects).

Based on the cross referenced data, a cost range for the request may be determined in 410. The cost range may include, for example, the reimbursement amount, the time required to receive reimbursement, the terms of payment, the requested amount that is written off, the number of rounds of adjustments/negotiations, and/or other such aspects. Such cost range data may be referenced to other aspects. Based on such factors, a cost range may be determined for the procedure data. Such a cost range may be determined via, for example, a machine learning technique. The cost range may be communicated to a user device for display on a GUI of the user device.

Thus, based on technique 400, financial forecasting may be performed to allow for the determination of the likely payout amount of procedures performed or procedures that are currently being performed. It is appreciated that the standardization of procedure data may allow for the accurate determination of the cost range. Otherwise, disparate procedure data shapes may cause inaccurate forecasting due to misinterpreted data.

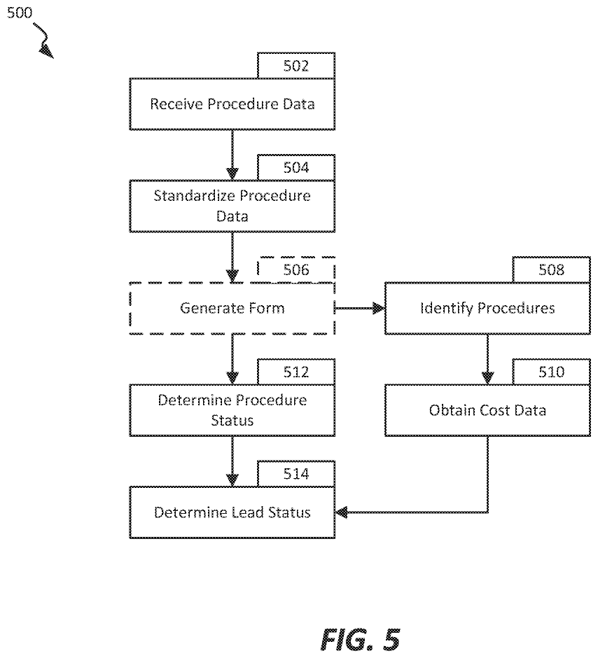

FIG. 5 illustrates technique 500, which may be a technique for determining the status of a current lead. In 502, platform 102 may receive procedure data. The procedure data may be data associated with procedures that have been performed, are currently being performed, are scheduled to be performed, and/or may possibly be performed (e.g., a lead). Such procedure data may be as described herein and may, in certain embodiments, be of a non-standard format.

In 504, the procedure data may be standardized. Thus, for example, the procedure data may first be parsed to determine various aspects (e.g., details of the procedure) within the procedure data. The aspects may then be contained within procedure data of a standardized format (e.g., a standardized table or another data format). The standardized format may be configured such that data elements may each contain certain data aspects and such data aspects may be determined and populated within the standardized format from the procedure data and/or from platform 102 accessing various databases and identifying such data (e.g., the identification number associated with a practitioner). In certain embodiments, data that is not used for identify procedures in 508 and/or determine procedure status in 512 may be anonymized or encrypted as part of the standardization process and/or later in the process. Such anonymization or encryption may be according to techniques described herein.

In optional 506, a form may be generated according to the techniques described herein (e.g., the technique described in FIG. 3). The form and/or the various form sections of the form may allow for the identification of the procedures in 508 and/or the procedure status in 512 (e.g., based on the data within the form, the form sections and the data that those form sections are configured to obtain, and/or based on other aspects of the form and/or form sections). The form and/or the form sections may then allow for a determination of the procedures and/or procedure status. Thus, for example, the various form sections of the form may include a form section identifying the post-treatment status of a patient. Such a form section may allow for the determination of the procedure status (e.g., post-treatment).

In 512, the procedure status of the procedure(s) associated with the procedure data may be determined. The procedure status may be, for example, an indication of whether the procedure has been performed, is being performed, is to be performed, or may possibly be performed. In certain embodiments, determining the procedure status may be determined from the procedure data received. Alternatively or additionally, the procedure data may identify the specific procedure and the appropriate database may be accessed to determine the status.

Additionally, based on the procedure data, the identity of the procedures may be identified in 508. Procedures that are identified may include, for example, procedures that have been performed, are currently being performed, and/or are scheduled to be performed. In various embodiments, the data contained within the procedure data may allow for the categorization of the status of the procedures (e.g., performed, being performed, to be performed, or may possibly be performed). Thus, 508 may also perform the step of 512. Other embodiments may separate the identity of the procedure and the status of the procedure (e.g., the identity and status of the procedure may be independently indicated within the procedure data and/or within a database with separate data elements).

Based on the identity of the procedure(s), the cost data associated with the procedure may be obtained in 510. The cost data may be obtained similar to, for example, the cost data obtained in 408. The cost data may allow for the determination of a likely payout amount of procedures performed, planned, or possibly to be performed. Based on the cost data and the procedure status, the status of a current lead may be determined in 514. The status of the current lead may include, for example, the current procedure status and the likelihood of future compensation for the procedures. Based on the combination of the status and the likelihood of future compensation, a determination may be made as to, for example, the likelihood of payout or profit from the procedure. The status may be accordingly communicated to a user device for display on a GUI of the user device.

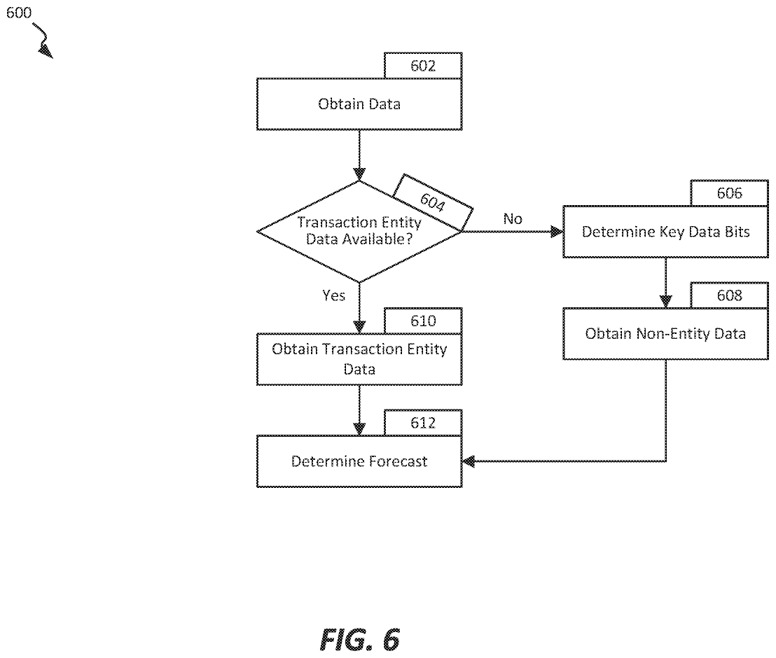

FIG. 6 illustrates technique 600, which may be a technique for forecasting of procedures based on transaction entity data. Such forecasting may be a forecasting of current opportunities or other such aspects, as described herein. Transaction entity data may be historic transaction data associated with an entity (“transaction entity”), such as the user, service provider (e.g., doctor or hospital), service receiver, and/or other such entity. Transaction entity data may allow for the determination of past and future patterns, trends, and other aspects of requests of a transaction entity.

In 602, data may be obtained (e.g., data may be received from a GUI of a user device or one or more databases may be accessed for the data). Such data may be various data as described herein, such as procedure data. Alternatively or additionally, the data may include data stored within databases of platform 102 and/or in areas accessible to platform 102 or received from user device 140 and/or an external entity 142. In a certain embodiment, the data may be associated with a request and platform 102 may identify the various aspects of the request and obtain such data or obtain additional data to the data received.

Based on the data received or accessed, platform 102 may determine whether transaction entity data is available within the data obtained and/or is stored within one or more databases within platform 102, in 604. Transaction entity data may include data generated by the entity (e.g., requesting entity) as well as data determined by platform 102 that is associated with the entity (e.g., determined from data communicated by the entity, by a vendor, and/or from publicly available data). In 604, platform 102 may determine whether transaction entity data is stored within one or more databases of platform 102. Such a determination may be performed by, for example, searching of the appropriate databases of platform 102 according to various techniques.

In certain embodiments, the search query for the databases may be based on the data obtained. Thus, for example, the data received may include data directed to various aspects of a request, such as the requesting entity, the procedure, the vendor that the request is provided to, and/or other such data. The search query may be based on such data received.

If transaction entity data is available, database(s) containing the transaction entity data may be accessed in 610 and the transaction entity data may be obtained. Platform 102 may, thus, access one or more database(s) containing the transaction entity data and pull from data from the database(s).

Based on such transaction entity data, the appropriate forecast performed with the transaction entity data in 612. For example, compensation data for a vendor may allow for the forecasting of the likely payout of a request with the vendor. The forecast of 612 may be communicated to a GUI of a user device.

If transaction entity data (e.g., data that identifies or is associated with the requesting entity) is not available, key data bits of the request may be determined in 606. Such key data bits may include portions of the data that is associated with key parts of the request. For example, the service provided, the time of service performed, the location of service performed, the governing regulatory bodies, the service receiver, the vendor (e.g., insurance entity), the requested compensation amount, and/or other such aspects may be determined. In certain embodiments, the key data bits may be determined through machine learning (e.g., determined by machine learning identifying which data bits allow for the determination of which data bits allow for accurate forecasting), through preprogrammed identifications, and/or through other determinations. In various embodiments, identification of the key data bits may include identifying that such data is present within one or more databases of platform 102 (e.g., through a search of the databases). In certain embodiments, if a determination is made that transaction entity data is not available, the data received in 602 may be anonymized or encrypted while still allowing for key data bits to be obtained (e.g., the key data bits may not be anonymized or encrypted in order to allow for access to the appropriate data bits).

Based on the key data bits determined in 606, such data may be obtained from the database(s) that the data is stored within, in 608. Forecasts may be performed in 612 utilizing the key data bits (e.g., data that is not directly associated with the transaction entity, but may nonetheless be analogous). In various embodiments, the forecasting may, thus, utilize data that is analogous to the transaction entity data. In certain embodiments, utilization of such data may be adjusted to provide a more accurate forecast. Accordingly, if analogous data is associated with a requesting entity from a lower cost area, the forecast may be adjusted upward.

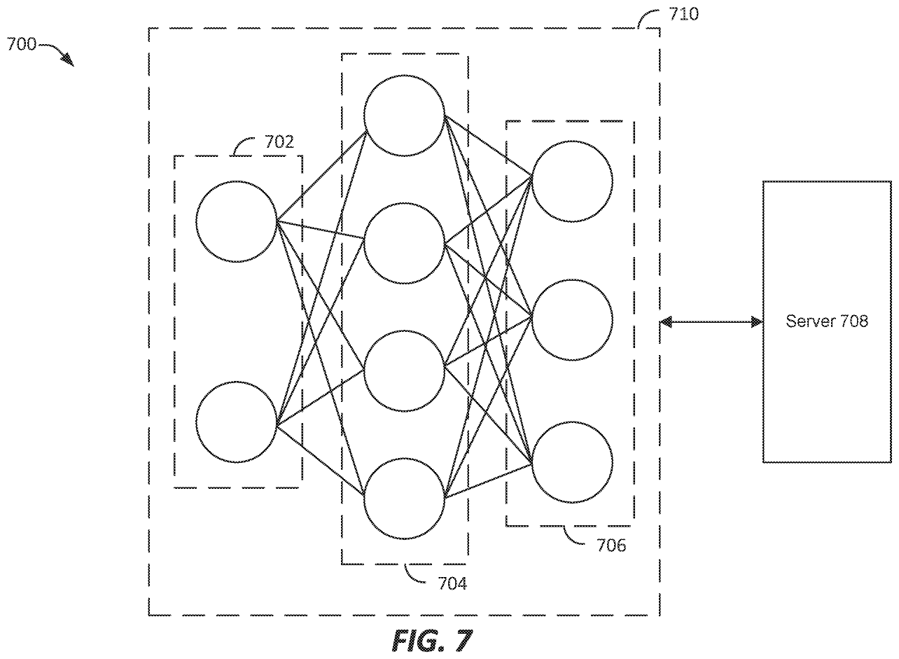

FIG. 7 illustrates an example of a neural network, configured in accordance with some embodiments. FIG. 7 illustrates system 700 that includes neural network 710 and server 708. Neural network 710 includes input layer 702, hidden layers 704, and output layer 706. Neural network 710 may be a machine learning network that may be trained to perform the techniques described herein (e.g., determine the form sections required for creation of a form, create the form, provide for forecasting, and/or other such techniques). Neural network may be communicatively coupled to server 708 to perform encryption/decryption as needed to provide data security. In various embodiments, neural network 710 may be one or a plurality of different neural networks, where each neural network is configured to create and/or provide updates to a machine learning network. Thus, system 700 may be a distributed machine learning system.

Neural network 710 may be trained with inputs. Input layer 702 may include such inputs. Such inputs may include, for example, various data stored within platform 102, as described herein. Hidden layers 704 may be one or more intermediate layers where logic is performed to determine various aspects of the data (e.g., standardization of various portions of data so that data not associated with the transaction entity may be utilized in forecasts). Output layer 706 may result from computation performed within hidden layers 704 and may output, form section determinations, forecasts, and/or other such aspects. Output layer 706 may, thus, provide one or more machine learning models and/or updates to machine learning models.

Machine learning may be utilized to determine parameters of the techniques described herein and/or to perform the techniques themselves. In various embodiments, machine learning may continuously or periodically refine the determinations based on data received (e.g., additional jurisdiction data received).

As the data utilized to train a machine learning model may include sensitive information, encryption/decryption may be performed to protect sensitive information, while still allowing for training of the machine learning model on the data. System 700 may be configured to perform, for example, homomorphic encryption on the sensitive data. In certain embodiments, system 700 may be configured to perform encryption and decryption operations within neural network 710, homomorphic operations within server 708, and authentication operations (e.g., the generation of keys for authentication purposes).

In various embodiments, outputs from neural network 710 may, instead of directly updating a machine learning model, homomorphically encrypt the outputs and provide the outputs to server 708. Server 708 may be configured to receive outputs from various neural networks, integrate such outputs into one or more models, and provide the results from such integration to the various neural networks.

Thus, upon receipt of outputs from various neural networks, server 708 may integrate the homomorphically encrypted outputs into an updated output package, which is then provided to the various neural networks, including neural network 710. Upon receipt of the updated output package, neural network 710 may then decrypt the package and update the machine learning model. Such a technique allows for the machine learning model of each neural network to include sensitive data of other networks implicitly and, thus does not expose sensitive data of one network to other networks. As such, a plurality of neural networks may update and refine the machine learning model without sensitive data being exposed.

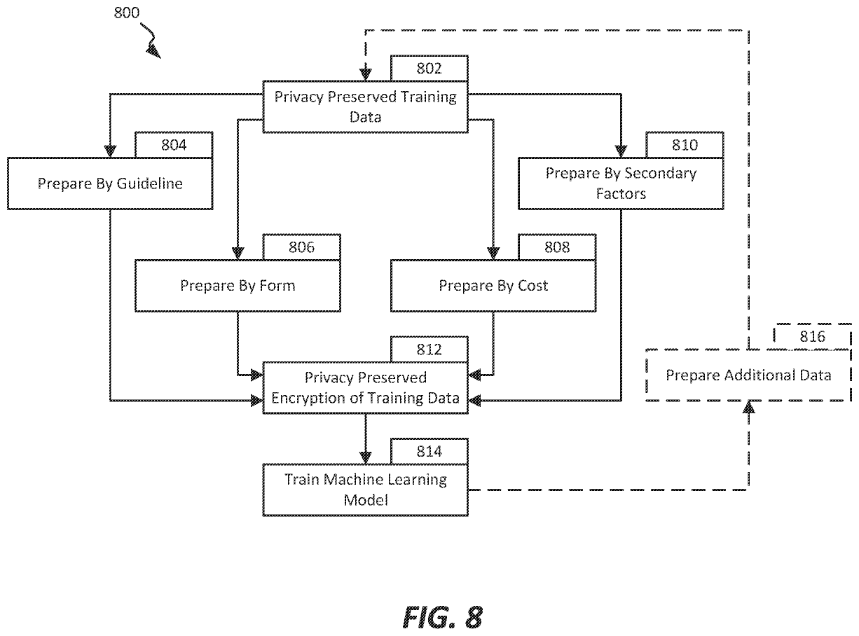

FIG. 8 illustrates a machine learning billing data training technique, in accordance with some embodiments. FIG. 8 illustrates technique 800 for training a machine learning model to perform techniques described herein.

In 802, training data may be received or prepared from standardized procedure data. The training data may be actual data (e.g., real world data), data created specifically for training (e.g., due to privacy laws), anonymized data (e.g., data where the privacy of the organizations or individuals that the data originated from is preserved), and/or data encrypted using homomorphic encryption to preserve the privacy of individuals represented in the data without skewing the results. Variously, the training data may be data related to one or more requests and the responses by vendors to the requests. The training data may include data directed to a plurality of aspects of the requests, such as the requesting entity, the vendor, the procedure performed, the details of the procedure, the response (e.g., compensation amount), the time for response, and/or other such datapoints as described herein.

Anonymization may include, for example, blurring specific values (e.g., numerical values, names, voices, videos, and/or other such data) of the data. Such blurring may include, for example, changing a specific instance to a value range. In certain embodiments, anonymization may be informed by the machine learning model. That is, the machine learning model may determine appropriate ranges for blurring.

In various embodiments, the training data may be sorted according to various categories of the data, as detailed in 804-810. The sorted training data may then be utilized to train a machine learning model in 814. Such training may include training by each set of data prepared in 804-810 (e.g., based on the categories of the data prepared). The machine learning model may be trained to, for example, determine any patterns or other correlations between the aspects of the training data and, for example, the responses to the request (e.g., the amount of compensation provided and/or the time required to respond to the request).

As an example of preparation of data, in 804, the data elements of the training data may be sorted according to guidelines. Thus, the requests may be sorted according to the guidelines (e.g., law or preferences of the vendor) that the requests were provided within. Such sorting may allow for the determination of whether patterns are present in requests made in certain guidelines. Such determinations may, for example, provide for more accurate forecasts of requests that are made in such guidelines by the machine learning model.

In 806, the training data may be sorted by forms. Such sorting may include categorizing based on the substantive (e.g., inquiries) of the forms. Thus, requests that utilize the same or similar forms (e.g., forms of the same or similar form sections) may be grouped accordingly. Accordingly, the sorting in 806 may be sorting based on the make-up of the forms of the request. Training of the machine learning based on the anonymized forms may allow for the determination of patterns based on forms. Such determinations may allow for more accurate forecasting based on the type of form created for a request (e.g., which form sections are used in the form for the request).

In 808, the training data may be sorted according to costs of the request. Accordingly, high value requests may be sorted together (and low value requests may be sorted together as well). Training of the machine learning model based on the costs of the requests may allow for the determination of patterns based on costs of the requests. For example, the compensation provided by a vendor may be influenced by the cost of the request.

In 810, the training data may be sorted by other secondary factors (e.g., the identity of the vendor, the geographical location that the request originates from, details of the procedure performed, and/or other such aspects). Training of the machine learning based on such factors may allow for the determination of patterns within the factors.

In various embodiments, the preparation of 804-810 may be augmentation of data. That is, the original structure of the data may be preserved, but may be augmented with the preparation of the respective category (e.g., through the additional of an additional data element to the data package as a whole or to each individual data element). Such augmented data may be used for training.

Based on such preparation, the training data may be encrypted in 812. Encryption of the training data may be via, for example, homomorphic encryption to preserve the privacy of individuals represented in the data without skewing the results. Thus, in a certain embodiment, data may be anonymized in 802 and encrypted in 812. The machine learning model may then be trained in 814 using the encrypted data.

In optional 816, additional data may be prepared to further train the machine learning model. Additional data may be any type of data as described herein. The additional data may allow for further rounds of training of the machine learning model.

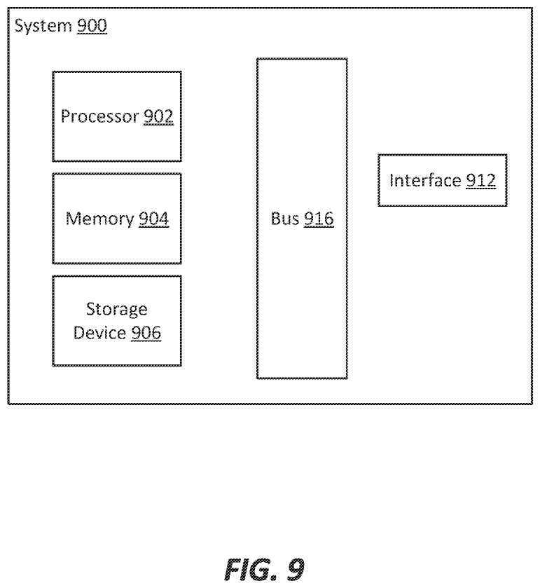

FIG. 9 illustrates a block diagram of an example computing system, in accordance with some embodiments. According to various embodiments, a system 900 suitable for implementing embodiments described herein includes a processor 902, a memory module 904, a storage device 906, an interface 912, and a bus 916 (e.g., a PCI bus or other interconnection fabric.) System 900 may operate as variety of devices such as a server system such as an application server and a database server, a client system such as a laptop, desktop, smartphone, tablet, wearable device, set top box, etc., or any other device or service described herein.

Although a particular configuration is described, a variety of alternative configurations are possible. The processor 902 may perform operations such as those described herein. Instructions for performing such operations may be embodied in the memory 904, on one or more non-transitory computer readable media, or on some other storage device. Various specially configured devices can also be used in place of or in addition to the processor 902. The interface 912 may be configured to send and receive data packets over a network. Examples of supported interfaces include, but are not limited to: Ethernet, fast Ethernet, Gigabit Ethernet, frame relay, cable, digital subscriber line (DSL), token ring, Asynchronous Transfer Mode (ATM), High-Speed Serial Interface (HSSI), and Fiber Distributed Data Interface (FDDI). These interfaces may include ports appropriate for communication with the appropriate media. They may also include an independent processor and/or volatile RAM. A computer system or computing device may include or communicate with a monitor, printer, or other suitable display for providing any of the results mentioned herein to a user.

Any of the disclosed embodiments may be embodied in various types of hardware, software, firmware, computer readable media, and combinations thereof. For example, some techniques disclosed herein may be implemented, at least in part, by non-transitory computer-readable media that include program instructions, state information, etc., for configuring a computing system to perform various services and operations described herein. Examples of program instructions include both machine code, such as produced by a compiler, and higher-level code that may be executed via an interpreter. Instructions may be embodied in any suitable language such as, for example, Java, Python, C++, C, HTML, any other markup language, JavaScript, ActiveX, VBScript, or Perl. Examples of non-transitory computer-readable media include, but are not limited to: magnetic media such as hard disks and magnetic tape; optical media such as flash memory, compact disk (CD) or digital versatile disk (DVD); magneto-optical media; and other hardware devices such as read-only memory (“ROM”) devices and random-access memory (“RAM”) devices. A non-transitory computer-readable medium may be any combination of such storage devices.

In the foregoing specification, various techniques and mechanisms may have been described in singular form for clarity. However, it should be noted that some embodiments include multiple iterations of a technique or multiple instantiations of a mechanism unless otherwise noted. For example, a system uses a processor in a variety of contexts but can use multiple processors while remaining within the scope of the present disclosure unless otherwise noted. Similarly, various techniques and mechanisms may have been described as including a connection between two entities. However, a connection does not necessarily mean a direct, unimpeded connection, as a variety of other entities (e.g., bridges, controllers, gateways, etc.) may reside between the two entities.

In the foregoing specification, reference was made in detail to specific embodiments including one or more of the best modes contemplated by the inventors. While various embodiments have been described herein, it should be understood that they have been presented by way of example only, and not limitation. For example, some techniques and mechanisms are described herein in the context of fulfillment. However, the disclosed techniques apply to a wide variety of circumstances. Particular embodiments may be implemented without some or all of the specific details described herein. In other instances, well known process operations have not been described in detail in order not to unnecessarily obscure the techniques disclosed herein. Accordingly, the breadth and scope of the present application should not be limited by any of the embodiments described herein, but should be defined only in accordance with the claims and their equivalents.

1. A method comprising:
receiving non-standardized medical procedure data from a graphical user interface (GUI) of a user device;
standardizing the medical procedure data, wherein the standardizing comprises:
parsing the non-standardized medical procedure data to determine procedure data details; and
creating standardized medical procedure data, the standardized medical procedure data comprising a standard data structure, wherein the parsed procedure data details are populated within the standard data structure;
determining, based on the standardized medical procedure data, procedure details;
determining cost data elements based on the procedure data;
accessing a cost database to obtain the cost data indicating the cost data elements;
forecasting a cost range based on the cost data elements; and
communicating the cost range for display on the GUI of the user device.

2. The method of claim 1, further comprising:
generating requirements data based on the medical procedure details;
obtaining, based on the requirements data, medical billing form section data associated with a plurality of form sections; and
combining the medical billing form section data to create a medical billing form, wherein the medical procedure details are determined, at least in part, from the medical billing form section data.

3. The method of claim 2, wherein the non-standardized medical procedure data comprises a request for the medical billing form, the medical billing form configured to obtain data in accordance with the requirements of a vendor.

4. The method of claim 3, wherein the requirements data comprises identification of the data to be obtained in accordance with the requirements of the vendor.

5. The method of claim 4, wherein the medical billing form section data, when combined into the medical billing form, is configured to allow for input of the data to be obtained in accordance with the requirements of the vendor.

6. The method of claim 1, wherein the cost database is configured to store medical cost data categorized based on one or more of a vendor, request, reimbursement amount, and/or reimbursement timeframe, and wherein the medical cost data comprises one or more of vendor responses to requests, an average or range of reimbursement provided by vendors, and/or an average or range of time required to provide reimbursement in response to requests.

7. The method of claim 1, wherein the standardizing the medical procedure data further comprises:
anonymizing the medical procedure data.

8. The method of claim 1, further comprising:
determining a medical procedure status associated with the medical procedure data.

9. The method of claim 8, further comprising:
determining, based on the cost data and the medical procedure status, a lead status, wherein the lead status indicates a likelihood of payout and/or a payout lead time.

10. The method of claim 1, wherein the procedure data comprises a request, and the method further comprises:
determining that transaction entity data is unavailable, wherein the transaction entity data comprises historical data generated by an entity associated with the medical procedure data;
determining key data bits associated with portions of the request;
obtaining non-entity data based on the key data bits; and
determining a forecast based on the non-entity data.

11. A system comprising:
a plurality of databases;
a memory; and
a processor, configured to receive instructions from the memory and perform operations comprising:
receiving non-standardized medical procedure data from a graphical user interface (GUI) of a user device;
standardizing the medical procedure data, wherein the standardizing comprises:
parsing the non-standardized medical procedure data to determine procedure data details; and
creating standardized medical procedure data, the standardized medical procedure data comprising a standard data structure, wherein the parsed procedure data details are populated within the standard data structure;
determining, based on the standardized medical procedure data, procedure details;
determining cost data elements based on the procedure data;
accessing a cost database of the plurality of databases to obtain the cost data indicating the cost data elements;
forecasting a cost range based on the cost data elements; and
communicating the cost range for display on the GUI of the user device.

12. The system of claim 11, wherein the operations further comprise:
generating requirements data based on the medical procedure details;
obtaining, based on the requirements data, medical billing form section data associated with a plurality of form sections; and
combining the medical billing form section data to create a medical billing form, wherein the medical procedure details are determined, at least in part, from the medical billing form section data.

13. The system of claim 12, wherein the non-standardized medical procedure data comprises a request for the medical billing form, the medical billing form configured to obtain data in accordance with the requirements of a vendor.

14. The system of claim 13, wherein the requirements data comprises identification of the data to be obtained in accordance with the requirements of the vendor, and wherein the medical billing form section data, when combined into the medical billing form, is configured to allow for input of the data to be obtained in accordance with the requirements of the vendor.

15. The system of claim 11, wherein the non-standardized procedure data comprises audio, video, and/or images.

16. The system of claim 11, wherein the cost database is configured to store medical cost data categorized based on one or more of a vendor, request, reimbursement amount, and/or reimbursement timeframe, and wherein the medical cost data comprises one or more of vendor responses to requests, an average or range of reimbursement provided by vendors, and/or an average or range of time required to provide reimbursement in response to requests.

17. The system of claim 11, wherein the standardizing the medical procedure data further comprises:
anonymizing the medical procedure data.

18. The system of claim 11, wherein the operations further comprise:
determining a medical procedure status associated with the medical procedure data.

19. The system of claim 18, wherein the operations further comprise:
determining, based on the cost data and the medical procedure status, a lead status, wherein the lead status indicates a likelihood of payout and/or a payout lead time.

20. The system of claim 11, wherein the procedure data comprises a request, and the operations further comprise:
determining that transaction entity data is unavailable, wherein the transaction entity data comprises historical data generated by an entity associated with the medical procedure data;
determining key data bits associated with portions of the request;
obtaining non-entity data based on the key data bits; and
determining a forecast based on the non-entity data.
